(19) United States
(12) Patent Application Publication
(10) Pub. No.: US 2023/0021946 A1
(43) Pub. Date: Jan. 26, 2023

(54) MONOMERIC FUSION PEPTIDES AND METHOD OF USE THEREOF

(71) Applicant: ALLYSTA PHARMACEUTICALS, INC., Bellevue, WA (US)

(72) Inventors: Henry Hsu, Bellevue, WA (US); Laszlo Otvos, Audubon, PA (US)

(21) Appl. No.: 17/854,594

(22) Filed: Jun. 30, 2022

Related U.S. Application Data
(60) Provisional application No. 63/216,688, filed on Jun. 30, 2021.

(51) Int. Cl. C07K 14/605; A61P 3/10; A61K 47/64

(52) U.S. Cl. CPC C07K 14/605; A61P 3/10; A61K 47/64

(57) ABSTRACT

A fusion peptide comprising a GLP1 variant, and at least one adiponectin agonist peptide which is chemically attached to the GLP1 variant via by a spacer. The GLP1 variant portion can include one or more substitutions relative to the native GLP1. The adiponectin agonist peptide can be attached to the GLP1 variant at different attachment sites. A method of treating a metabolic disorder or condition using the fusion peptide is also provided.

### FIELD OF INVENTION

The invention relates to monomeric peptides with dual agonist activity, in particular, peptides that comprise modified glucagon-like 1 agonist and an adiponectin receptor agonist, and their use in treatment of diabetes.

### BACKGROUND

Type 2 diabetes mellitus poses a serious threat to the public health worldwide. Currently, most available treatment regimens for metabolic diseases such as diabetes target single aspects, such as enhancing insulin production. However, resistance to energy homeostatic perturbations, combined with the heterogeneous pathophysiology of human metabolic disorders, has limited the sustainability and efficacy of current pharmacological options. Emerging insights into the cellular features of dysregulated energy expenditure, and insulin resistance suggest that coordinated targeting of multiple signaling pathways is probably necessary for sizeable improvements to reverse the progression of these diseases beyond targeting only glucose dynamics.

Glucagon Like Peptide-1, also referred to as GLP1 or GLP1(7-36) amide herein, having the amino acid sequence [free amino terminus-HAEGTFTSDVSSYLEGQAAKEFIAWLVKGR-amide (SEQ ID NO:1)], is a 30 amino acid residue hormone which regulates glucose homeostasis by controlling the release of insulin from the beta cell of pancreas following food ingestion. The hormone is released from the gastrointestinal tract following nutrient/food consumption and stimulates the acute release of insulin postprandially to regulate blood glucose. Additionally, GLP1 slows digestive action in the gastrointestinal tract by acting as a satiety factor and reducing the amount of food intake by delaying the time for emptying digested food in the gastrointestinal tract and can reduce body weight. Peptide drugs with GLP1 agonist activity have been modified to reduce proteolytic degradation, particularly by dipeptidyl peptidase 4 (DPP-4) and prolong half-life. However, GLP1 analogues, by themselves, do not have anti-inflammatory or anti-fibrotic actions and do not affect insulin resistance in target tissues.

The combination of GLP1 with hormone analogues have been described and includes GLP1 in combination with cholecystokinin, peptide YY, glucagon, GLP2, gastric inhibitory polypeptide (GIP), gastrin, neurotensin, fibroblast growth factor 21 (FGF21), melanocortin receptor 4 (MC4R) agonists, insulin and SGLT2 inhibitors.

As the most abundant peptide secreted by adipocytes, adiponectin is a key regulator of the interrelationship between adiposity, insulin resistance and inflammation. Central obesity accompanied by insulin resistance is a key factor in the development progression to Type 2 diabetes and its complications.

Adiponectin is present in the circulation as a monomer, trimer, hexamer, or protein aggregate of high molecular weight complexes up to 18-mers. AdipoR1 and AdipoR2 are its major receptors in mediating in vivo and cellular actions. Adiponectin receptors signal AMP (adenosine mono phosphate) kinase (AMPK) activation, exerting direct effects to regulate energy homeostasis within multiple organs, including adipose tissue, muscles, liver, and pancreas to improve insulin sensitivity. These receptors are ubiquitously expressed on virtually all tissues and cell types. In addition, activation of adiponectin receptor signaling affects multiple intracellular signaling pathways, leading to a wide range of beneficial actions, including: inhibition of de novo lipogenesis and increased lipid oxidation in liver and muscle; decrease in inflammatory mediators including inhibition in inflammatory cytokines such as IL-6, TNF-alpha, and IL-1b as well as inhibition of monocyte activation; anti-apoptotic and cellular regenerative actions following injury; and inhibition of pro-fibrotic pathways.

A molecule or drug that includes the actions of GLP1 and adiponectin may provide benefits not seen with either when given alone. In particular, GLP1 analogues are effective in increasing post-prandial insulin production, while adiponectin is effective in increasing insulin sensitivity. The combination of these two actions results in greater effects on glucose handling. One approach to combining the actions of GLP1 and adiponectin that has been attempted is to create a fusion protein containing GLP1 with the globular adiponectin. Gao, et. al. designed the fusion protein based on the molecular properties of GLP1 and globular adiponectin (Mingming Gao, Yue Tong, Wen Li, Xiangdong Gao & Wenbing Yao (2013), Artificial Cells, Nanomedicine, and Biotechnology, 41:3, 159-164.) The plasmid construct was expressed in bacteria and the resulting large protein was extracted and purified and was shown to retain glucose-lowering activity. However, a limitation of this approach is the use of the costly and inefficient expression systems to produce a large protein that must be carefully processed to retain structural integrity.

Thus, the development of recombinant forms of adiponectin suitable for human administration has proved challenging due to its large size, extensive post-translational modifications, and tendency to self-aggregate, and expense associated with mammalian protein production systems. Another approach is the identification of smaller peptide analogues capable of binding to adiponectin receptors and demonstrating agonist activity. One such agonist peptide, a 10 amino acid chemically synthesized peptide called ALY688 (also known as ADP355) [(H-DAsn-Ile-Pro-Nva-Leu-Tyr-DSer-Phe-Ala-DSer-NH₂) (where H represents the free amino terminus D in italics shows that the given amino acid is of the D-configuration and NH₂ at the end shows that the carboxy terminus is amidated, and Nva refers to L-norvaline (SEQ ID NO:2)), appears to bind to and activate the adiponectin receptors in a specific manner.

### SUMMARY OF THE INVENTION

In one aspect, the present disclosure provides a fusion peptide comprising a GLP1 variant and at least one adiponectin agonist peptide, wherein the at least one adiponectin agonist peptide is chemically attached to the GLP1 variant via by a spacer. In some embodiments, the GLP1 variant comprises a substitution with Gly at position 8. In some embodiments, the GLP1 variant comprises a substitution with Lys at position 18. In some embodiments, the GLP1 variant comprises a substitution with Lys at position 22. In these embodiments, the position corresponds to the position of SEQ ID NO:1. In some embodiments, the GLP1 variant has a sequence selected from the group consisting of SEQ ID NO:4, SEQ ID NO:5, and SEQ ID NO:6.

In some embodiments, the at least one adiponectin agonist peptide is attached via the spacer at the position 26 of the GLP1 variant.

In some embodiments, the at least one adiponectin agonist peptide is attached via the spacer at the position 34 of the GLP1 variant.

In some embodiments, the at least one adiponectin agonist peptide comprises a first adiponectin agonist peptide and a second adiponectin agonist peptide, the first adiponectin agonist peptide and the second adiponectin agonist peptide being the same or different and each attached via a spacer at two different positions of the GLP1 variant. The different two attachment sites can comprise position 26 and position 34 of the GLP1 variant, wherein the positions correspond to the positions of SEQ ID NO:1.

In some embodiments, the at least one adiponectin agonist peptide comprises ALY688.

In some embodiments, the spacer comprises GGG.

In another aspect, the present disclosure provides a method of treating a patient having type 2 diabetes mellitus, the method comprising administering to the patient an effective amount of a fusion peptide disclosed herein.

### BRIEF DESCRIPTION OF THE DRAWINGS

FIGS. 1A-1E schematically show structures of certain fusion peptides according to some embodiments of the present disclosure.

FIGS. 2A-2C show GLP1 receptor activation by certain ALY688-GLP1v fusion peptides of the present disclosure in HEK-hGLP1R-Luc cells. HEK-hGLP1R-Luc cells were treated with 100 μl of either GLP1v with gly8 substitution (2A) to evaluate dose dependency of GLP1 receptor activation; or ALY688-GLP1v fusion peptides (ALY688 attached to residues 18, 22, 26, 34 of GLP1v) at concentrations of 50 nM (2B) and 100 nM (2C) to evaluate their activation of GLP1 receptor signaling (n=3).

FIG. 3 shows screening of time- and concentration-dependent adiponectin signaling effects of certain ALY688-GLP1v fusion peptides of the present disclosure in L6 skeletal muscle cells using pP38MAPK ELISA. L6 skeletal muscle cells were incubated with the four different ALY688-GLP1v fusion peptides (0, 100, 300, 500 nM), gAd (1 μm/ml), fAd (10 μm/ml), ALY688 (100 nM) and Anisomycin (0.2, 1 μm/mL) for 15 or 30 mins, followed by an assessment of adiponectin-like signaling via pP38MAPK ELISA.

FIG. 4 depicts a testing procedure used in an example of the present disclosure.

FIGS. 5A-5B show blood glucose before and after glucose load (5A) and AUC (5B) in an example of the present disclosure. *p<0.05, **p<0.01 and ***p<0.001 with a two-ways ANOVA and Bonferonni's post-test vehicle vs all the other groups. **p<0.01 with a Kruskal-Wallis and a Dunns post-test, vehicle vs all the other groups.

FIG. 6 shows pharmacokinetic profiles of GLP1 and certain fusion peptides of the present disclosure in mice.

### DESCRIPTION OF EMBODIMENTS OF THE INVENTION

The present invention provides fusion peptides formed by the conjugation of a first moiety, a GLP1 variant, with a second moiety, which is a short peptide-based adiponectin receptor agonist, by a suitable chemical linker or spacer.

Due to the differing mechanisms of action, the combination of GLP1 with adiponectin analogue is a novel approach towards enhancing a range of desirable effects in conditions where metabolic dysregulation is associated with inflammation and/or fibrosis. In Type 2 diabetes, GLP1 action to stimulate post-prandial insulin release coupled with adiponectin's action to improve insulin sensitivity in muscle and liver, would be a complementary approach to improving overall glycemic control.

As peptides generally require injection (e.g., intravenous or subcutaneous), it would be desirable to be able to minimize the number of injections of separate medications. Thus, a fusion peptide that preserves the activity of its components and could be formulated into a single injection would be preferable to administering two separate injections. Moreover, it is easier for a single fusion peptide to reach the target tissue than two peptides at the same time making a single chemical entity preferable to a physical mixture of two peptides in an inoculum. The fusion peptide can also have superior pharmacokinetic and stability properties compared with at least one of the individual peptides. Also, a single formulation can be superior to a composition formulation that contains two individual peptides, each with its own requirements for stability, such as pH, need for stabilizing excipients, and need for components to maintain solubility.

Integrating the active site of the adiponectin protein instead of full globular adiponectin (gAd) or full-length adiponectin (fAd) can provide a fully peptidic drug with all the advantages of ALY688 compared to gAd or fAd in a therapeutic setting. The fusion peptides described herein maintain the activity of its individual components, while broadening the overall action of the fusion peptide beyond the components.

The term “fusion peptide” used herein refers to a peptide, or a peptide derivative, containing at least two peptide portions that are fused or chemically conjugated together. The fusion peptide can take a branched arrangement with one or more branches. The term “peptide” used herein refers to two or more amino acids linked in a chain, preferably by amide bonds, but also refers to derivatives of such structure wherein certain natural amino acid residues are replaced by non-natural residues. The fusion peptide can be prepared by solid-phase or a combination of solid-phase and liquid peptide synthetic methods, and thus, the non-natural amino acids can be selected from those that are commercially available in forms ready for large scale peptide synthesis.

The term “GLP1 variant” (or “GLP1v”) used herein refers to a modified GLP1 in which one or more amino acids in the GLP1 are replaced/substituted by other amino acid(s) or chemicals such as lipids. The sites of such substitutions are numbered according to the positions in the sequence of the native GLP1 (SEQ ID NO:1), and the type of substitutions are also based on the GLP1. Such substitutions increase the therapeutic efficacy when used in a human or veterinary drug setting. GLP1 variants described in this application maintains the GLP1 functions based upon binding to the GLP1 receptor and the ability to reduce glucose levels in appropriate models.

In some embodiments, the GLP1 variant can include substitutions at residue 8, e.g., Ala8Gly, at residue 18, e.g., Ser18Lys, at residue 22, e.g., Gly22Lys, etc. Numerous additional modifications can be made.

The second moiety of the fusion peptide can be a small molecule adiponectin receptor agonist peptide, such as the 10-mer ALY688, or the full 18 residue active binding site of adiponectin protein [(amino acids 149-166, H-Lys-Phe-His-Cys-Asn-Ile-Pro-Gly-Leu-Tyr-Tyr-Phe-Ala-Tyr-His-Ile-Thr-Val-NH₂ (SEQ ID NO:3) (Otvos et al., BMC Biotechnol 11, 90 (2011)), or any fragment thereof, as well as fragments thereof with ALY688-like substitutions or substitutions with other non-natural amino acid residues.

The first and second peptide part of the fusion peptide are linked by the linker/spacer. For example, the second peptide part can be attached (e.g., at its N terminal or C terminal) to the first peptide part at a position along its length, e.g., 18, 22, 26, 34 of the GLP1 variant. Such sites on the first peptide part are also referred to the attachment sites. The attachment sites can coincide with the substitution sites and can also be different from the substitution sites.

The linker that links the first and second moieties does not interfere with the activities of the constituent first or second peptides. The spacer can be composed of a peptide or nonpeptide. For example, the spacer peptide can include β- or γ-turn forming residues to not force the conformation of the constituents into α-helices or β-pleated sheets. Three to four residue spacers made from glycines, prolines, serines or similar turn forming residues usually make suitable turns between the constituents of the fusion peptides.

Such fusion peptides can include the conjugation product of the first peptide with more than one, for example, 2, 3, 4, or even more molecules, of the second peptide, where each of the second peptide is attached to the GLP1 variant at a respective attachment site with a (same or different) chemical linker.

In one embodiment of the fusion peptides of the present disclosure, alanine at 8^{th} position (Ala⁸) of GLP1 is replaced with glycine (Gly⁸) (SEQ ID NO:4). It is noted Ala⁸ is a cleavage site of dipeptidyl peptidase-4 (DPP-IV) resulting in protein degradation, and the replacement of Ala⁸ with Gly⁸ results in prolonged half-life by preventing the cleavage and reduction in receptor binding efficacy.

In another embodiment, the serine at 18^{th} position of GLP1 is substituted with lysine (SEQ ID NO:5). The second moiety can be attached to the Lys¹⁸ via a spacer (e.g., -GGG- (or G3) spacer). Ser¹⁸ is a cleavage site of neutral endopeptidase 24.11 (NEP24.11), therefore the replacement of Ser¹⁸ with a bulky side-chain reduces the proteolytic degradation. Also, Ser¹⁸ does not play a role in receptor binding and activation, thus the replacement of Ser¹⁸ with Lys¹⁸ does not affect receptor binding efficacy.

In another embodiment, glycine at 22^{th} position (Gly²²) of GLP1 is replaced with lysine (Lys²²) (SEQ ID NO:6). The second moiety can be attached to the Lys²² via a spacer (e.g., G3 spacer). Gly²² is part of a dipeptide that connects the two helices in GLP1 and therefore does not have functional or structural significance. Thus, the replacement of Gly²² with lysine (Lys²²) does not affect receptor binding efficacy.

In one embodiment, Lys²⁶ is used as an attachment site of the GLP1 variant for attaching the second moiety. Lys²⁶ is two amino acids away from Phe²⁸ which is needed for receptor activation, therefore the attachment of a side chain at Lys²⁶ is not expected to interfere with receptor binding efficiency. Furthermore, in Liraglutide and Semiglutide, Lys²⁶ carries fatty acids and can be freely modified for different functional improvement without changes to the receptor binding capacity.

In another embodiment, Lys³⁴ is used as an attachment site for attaching the second moiety. Lys³⁴ is the last residue in the C-terminal helix of GLP1, and has no special function. Also, there is no negatively charged residue in 3-4 positions upstream, thus Lys³⁴ does not stabilize the helix through ionic interactions. Therefore, Lys³⁴ can be attached with a bulky side chain group without affecting the binding capacity of GLP1 variant.

In one embodiment, the fusion peptide takes the following form (SEQ ID NO:5 attached at Lys¹⁸ with -G3- spacer with ALY688):

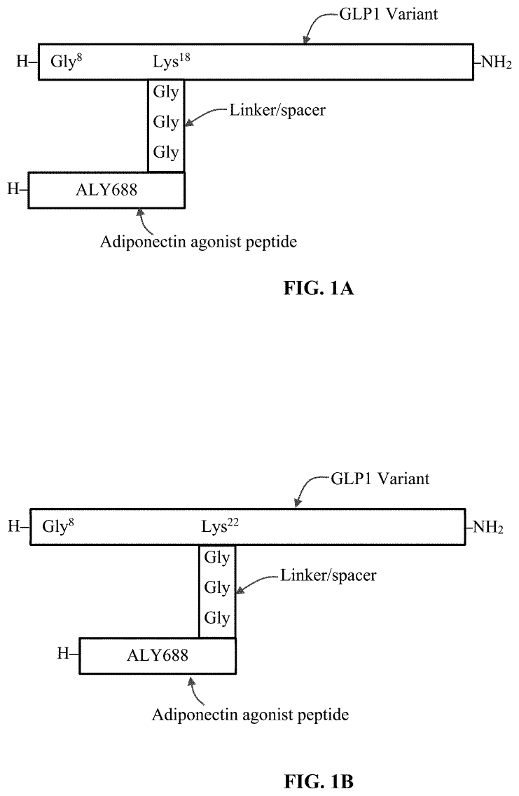

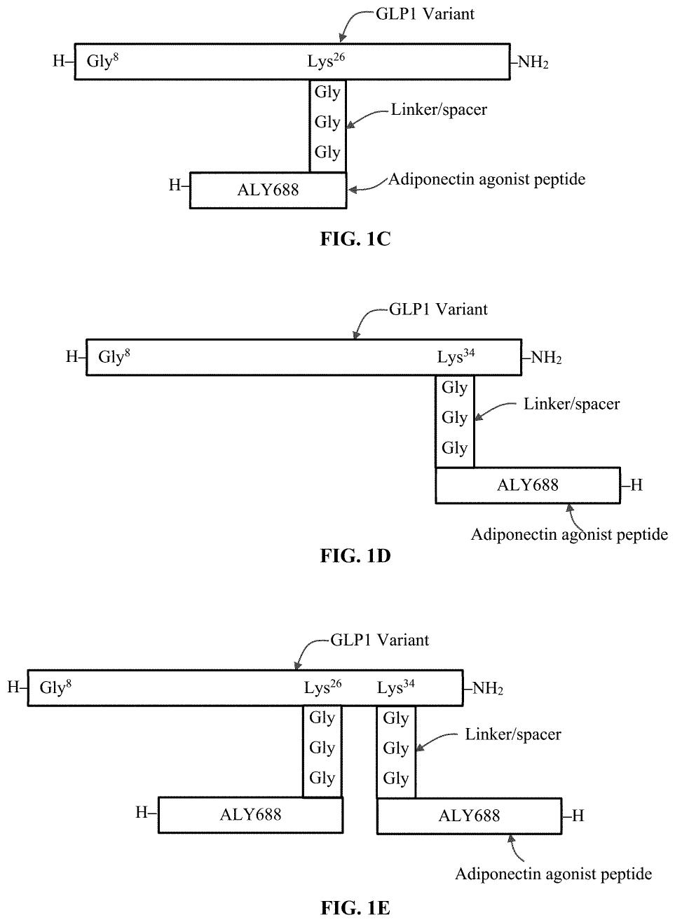

H-[GLP1-Gly⁸(7-36)Lys¹⁸]-(Gly-Gly-Gly-DSer-Ala-Phe-DSer-Tyr-Leu-Nva-Pro-Ile-DAsn-H)-NH₂. Its structure is illustrated in FIG. 1A.

In one embodiment, the fusion peptide takes the following form (SEQ ID NO:6 attached at Lys²² with -G3- spacer with ALY688):

H-[GLP1-Gly⁸(7-36)Lys²²]-(Gly-Gly-Gly-DSer-Ala-Phe-DSer-Tyr-Leu-Nva-Pro-Ile-DA sn-H)-NH₂. Its structure is illustrated in FIG. 1B.

In one embodiment, the fusion peptide takes the following form (SEQ ID NO:4 attached at Lys²⁶ with -G3- spacer with ALY688):

H-[GLP1-Gly⁸(7-36)Lys²⁶]-(Gly-Gly-Gly-DSer-Ala-Phe-DSer-Tyr-Leu-Nva-Pro-Ile-DA sn-H)-NH₂. Its structure is illustrated in FIG. 1C.

In one embodiment, the fusion peptide takes the following form (SEQ ID NO:4 attached at Lys³⁴ with -G3- spacer with ALY688):

H-[GLP1-Gly⁸(7-36)Lys³⁴]-(Gly-Gly-Gly-DSer-Ala-Phe-DSer-Tyr-Leu-Nva-Pro-Ile-DA sn-H)-NH₂. Its structure is illustrated in FIG. 1D.

In one embodiment, the fusion peptide takes the following form (SEQ ID NO:4 attached at Lys²⁶ and Lys³⁴ each with -G3- spacer with ALY688). Its structure is illustrated in FIG. 1E.

In a further aspect, the present disclosure provides a pharmaceutical composition comprising the fusion peptide described herein and a pharmaceutically acceptable carrier.

In a further aspect, the present disclosure provides a method of preventing, treating, or ameliorating Type 2 diabetes mellitus by administering a composition comprising a therapeutically effective amount of the fusion peptide described herein to a subject (e.g., a human patient).

### EXAMPLES

### Example 1. Stability Study of ALY688-GLP1v Fusion Peptides in Human Plasma

The stability and resistance to proteolytic degradation of a number of ALY688-GLP1v fusion peptides were assessed following incubation in human plasma at 37° C.

GLP1 peptide or ALY688-GLP1v fusion peptide (N=3/time point) was incubated in human plasma with K2EDTA anti-coagulant for the indicated times and then analyzed for residual content. HPLC (Supelco Discovery BIO Wide Pore C5-3 (2.1×50 mm) with a slow gradient to separate possible degradation products) was used to quantify each peptide. High resolution mass spectrometry (Thermo Q Exactive) Plus was used to collect full scan and MS2 spectra to characterize any possible degradants. The data were evaluated both manually and using Proteome Discoverer data mining software.

TABLE 1

| | | GLP1v Peptide | | |
|---|---|---|---|---|
| Time | Response | | | % of |
| (hrs) | Area | Mean | Std Dev | Time 0 |
| 0.00 | 7,793,534 | 8,553,114 | 573,466 | 100 |
| 0.00 | 8,686,788 | | | |
| 0.00 | 9,179,020 | | | |
| 1.00 | 7,442,318 | 7,292,410 | 185,441 | 85 |
| 1.00 | 7,031,101 | | | |
| 1.00 | 7,403,812 | | | |
| 8.00 | 5,007,493 | 3,494,879 | 1,151,774 | 41 |
| 8.00 | 3,261,903 | | | |
| 8.00 | 2,215,242 | | | |
| 22.00 | 2,100,712 | 2,094,668 | 15,037 | 24 |
| 22.00 | 2,109,303 | | | |
| 22.00 | 2,073,988 | | | |

TABLE 2

| | | ALY688-GLP1v_{(LYS26)} | | |
|---|---|---|---|---|
| Time | Response | | | % of |
| (hrs) | Area | Mean | Std Dev | Time 0 |
| 0.00 | 11,283,498 | 10,533,523 | 1,458,797 | 100 |
| 0.00 | 8,494,117 | | | |
| 0.00 | 11,822,953 | | | |
| 1.00 | 9,887,900 | 10,390,392 | 399,143 | 99 |
| 1.00 | 10,418,931 | | | |
| 1.00 | 10,864,345 | | | |
| 8.00 | 7,424,321 | 7,807,537 | 271,112 | 74 |
| 8.00 | 7,988,576 | | | |
| 8.00 | 8,009,713 | | | |
| 22.00 | 4,818,782 | 4,196,958 | 595,883 | 40 |
| 22.00 | 3,393,486 | | | |
| 22.00 | 4,378,606 | | | |

TABLE 3

| | | ALY688-GLP1v_{(LYS34)} | | |
|---|---|---|---|---|
| Time | Response | | | % of |
| (hrs) | Area | Mean | Std Dev | Time 0 |
| 0.00 | 17,842,282 | 16,089,042 | 2,447,925 | 100 |
| 0.00 | 17,797,594 | | | |
| 0.00 | 12,627,250 | | | |
| 1.00 | 14,976,839 | 15,289,758 | 1,868,882 | 95 |
| 1.00 | 13,173,412 | | | |
| 1.00 | 17,719,021 | | | |
| 8.00 | 12,992,258 | 11,836,948 | 879,750 | 74 |
| 8.00 | 10,859,437 | | | |
| 8.00 | 11,659,150 | | | |
| 22.00 | 9,036,370 | 7,922,840 | 957,088 | 49 |
| 22.00 | 6,699,694 | | | |
| 22.00 | 8,032,456 | | | |

In the above tables, Temperature=37° C., Concentration=1000 ng/ml, and N=3/time point.

Results: As shown in the above Tables 1-3, ALY688-GLP1v_{(Lys26)} and ALY688-GLP1v_{(Lys34)} where the GLP1v contains Gly8 substitution relative to GLP1 (when referring to a specific ALY688-GLP1v fusion peptide, the subscript at the end is used to indicate the attachment site) fusion peptides provides better resistance to proteolytic degradation when incubated at 37° C. in human plasma, as shown by the higher proportion of intact peptide compared with GLP1 peptide following 8 and 22 hours of incubation.

### Example 2. In Vitro Analysis of GLP1 Receptor Activation by ALY688-GLP1v Fusion Peptides

To determine if one or more of the ALY688-GLP1v fusion peptides retained its ability to activate the GLP1 receptor, an in vitro reporter cell line was used to quantitate the level of GLP1 activation. The activation of GLP1 receptors was evaluated using GLP1 itself and four different ALY688-GLP1v fusion peptides to determine if the fusion peptides retained their ability for GLP1 receptor activation. In these four different fusion peptides, ALY688 attached to residues 18, 22, 26, 34 on GLP1v sequences, respectively, and all GLP1v portion has Gly8 substitution. When ALY688 is attached to residue 18, residue 18 is substituted by Lys; when ALY688 is attached to residue 22, residue 22 is substituted by Lys; for ALY688 attached at positions 26 and 34 of GLP1v (which already has Lys residues), Ser18 and Gly22 on the GLP1v are not substituted. The sequences of these four ALY688-GLP1v fusion peptides are as follows:

ALY688-GLP1v_{(Lys18)} (or simply denoted as
“Lys 18”, or “Lysl8 sub” in the figures of
this disclosure):
H-His⁷-Gly-Glu-Gly-Thr-Phe-Thr-Ser-Asp-Val-Ser-
Lys¹⁸(Gly-Gly-Gly-DSer-Ala-Phe-DSer-Tyr-Leu-Nva-
Pro-Ile-DAsn-H)-Tyr-Leu-Glu-Gly-Gln-Ala-Ala-Lys-
Glu-Phe-Ile-Ala-Trp-Leu-Val-Lys-Gly-Arg³⁶-NH₂
ALY688-GLP1v_{(Lys22)}: (or simply denoted as
“Lys 22”, or “Lys22 sub” in the figures of
this disclosure)
H-His⁷-Gly-Glu-Gly-Thr-Phe-Thr-Ser-Asp-Val-Ser-
Ser-Tyr-Leu-Glu-Lys²²(Gly-Gly-Gly-DSer-Ala-Phe-
DSer-Tyr-Leu-Nva-Pro-Ile-DAsn-H)-Gln-Ala-Ala-Lys-
Glu-Phe-Ile-Ala-Trp-Leu-Val-Lys-Gly-Arg³⁶-NH₂
ALY688-GLP1v_{(Lys26)}: (or simply denoted as
“Lys 26”, or “Lys26 sub” in the figures of
this disclosure)
H-His⁷-Gly-Glu-Gly-Thr-Phe-Thr-Ser-Asp-Val-Ser-
Ser-Tyr-Leu-Glu-Gly-Gln-Ala-Ala-Lys²⁶(Gly-Gly-Gly-
DSer-Ala-Phe-DSer-Tyr-Leu-Nva-Pro-Ile-DAsn-H)-Glu-
Phe-Ile-Ala-Trp-Leu-Val-Lys-Gly-Arg³⁶-NH₂
ALY688-GLP1v_{(Lys34)}: (or simply denoted as
“Lys 34”, or “Lys34 sub” in the figures of
this disclosure)
H-His⁷-Gly-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Lys³⁴(Gly-Gly-Gly-DSer-Ala-Phe-
DSer-Tyr-Leu-Nva-Pro-Ile-DAsn-H)-Gly-Arg³⁶-NH₂

These sequences are also used in other examples herein.

A biosensor reporter cell model, HEK-hGLP1R-Luc, derived from human embryonic kidney 293 (HEK293) allowed for screening the activation of GLP1 receptor dependent signaling based upon the principle that the activation of the GLP1 receptor, or the GIP receptor respectively, leads to the production of cyclic adenosine monophosphate (cAMP) and the expression of luciferase gene, indicating activity of the ligand.

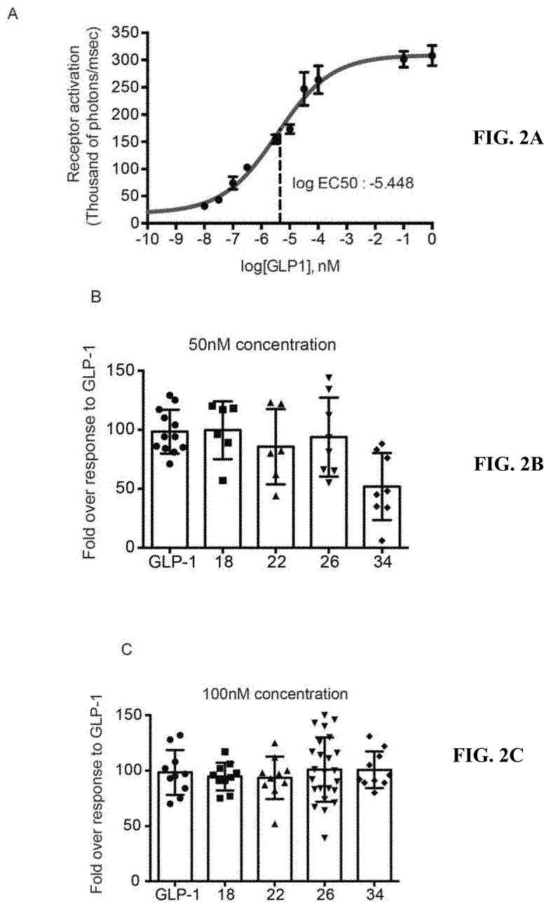

Results: as demonstrated in FIGS. 2A-2C, GLP1 showed a typical dose-dependent activation of luciferase activity (EC50 54 nM) in these cells which were engineered to biomark GLP1 receptor dependent signaling and 50 nM and 100 nM concentrations were chosen to test effects of ALY688-GLP1v fusion peptides (attachment of ALY688 at the positions of 18, 22, 26 and 34 of GLP1v, their structure having been described above). Each of the fusion peptides maintained the ability to activate the GLP1 receptor. Between the GLP1 standard and the different ALY688-GLP1v fusions, there was no significant difference in response, showing that the ability of the fusions to activate GLP1 was retained after attachment of the ALY688 peptide to GLP1v.

### Example 3: In Vitro Analysis of Adiponectin Signaling Effects of ALY688-GLP1v Fusion Peptides

ALY688 alone has been shown to induce adiponectin-like signaling in L6 murine skeletal muscle cells, including increased P38MAPK (T180/Y182) phosphorylation detected via ELISA analysis and confirmed by immunofluorescent imaging of phosphorylation-dependent translocation to the nucleus. P38MAPK is a known adiponectin-receptor signaling kinase involved in the beneficial metabolic effects of adiponectin. This was used to evaluate if the four different ALY688-GLP1v fusion peptides retain adiponectin-like signaling activity as previously shown with ALY688 alone.

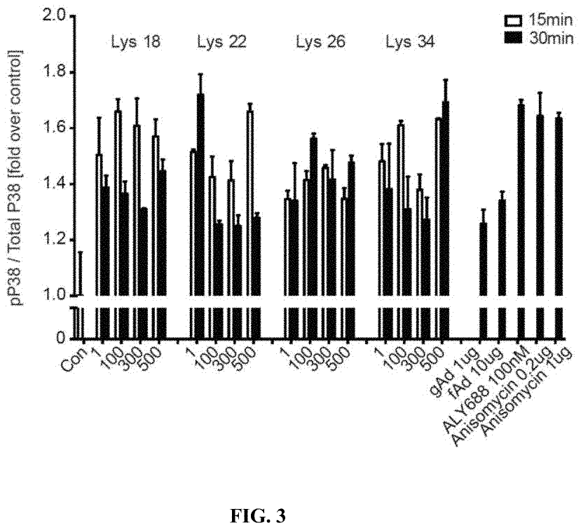

Results: Activation of p38MAPK by all four fusion peptides (same as those four fusion peptides in Example 2) were observed. As shown in FIG. 3, the activation of p38MAPK observed in response to fusions was similar to ALY688 alone and at least as potent as recombinant globular (gAd) or full-length (fAd) adiponectin protein. Thus, the ALY688-GLP1v fusion peptides retained their ability to activate adiponectin signaling similar to ALY688 alone.

### Example 4. Effects of Single Intravenous Injection on Blood Glucose Levels in Animal Models (Mouse)

In order to determine if the ALY688-GLP1v fusion peptide retained the physiologic actions of GLP1 in the whole organism, mice were administered a single dose of each peptide and blood glucose was assessed to evaluate the glucose-lowering activity induced by GLP1 activation.

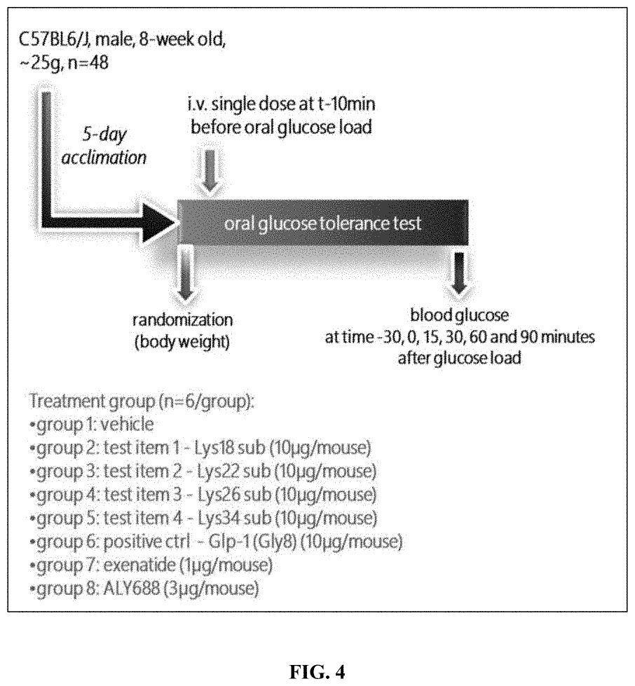

FIG. 4 shows schematically the grouping and test procedures, where mice were fasted for 4 hours and were then injected intravenously with vehicle, GLP1v (with Gly8 substitution) alone, GLP1v fusion peptides, exenatide or ALY688 at time 10 minutes before an oral glucose tolerance test. The oral glucose load was 1 g glucose/kg body weight. Blood glucose levels were measured with a glucometer at time −30 minutes, 0 (right before the oral glucose load), 15, 30, 60 and 90 minutes after the oral glucose load. Mice were euthanized after the time 90 minutes.

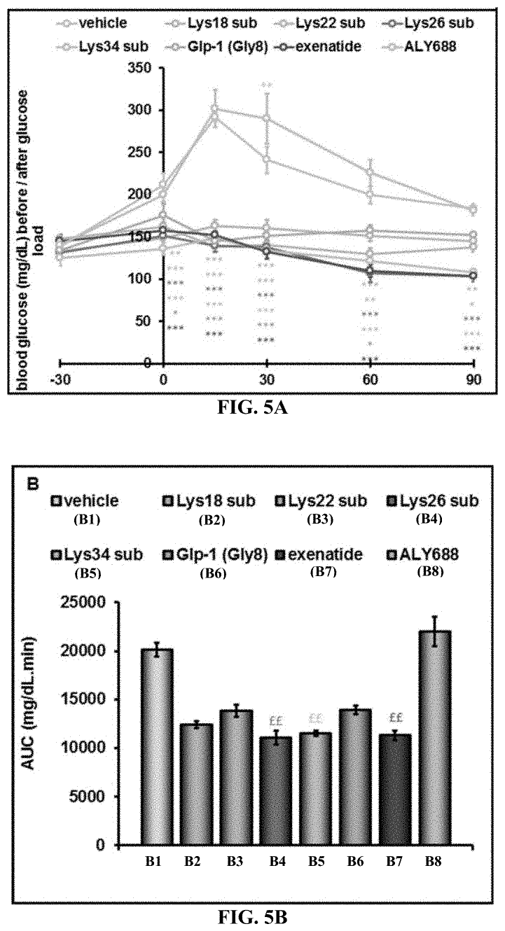

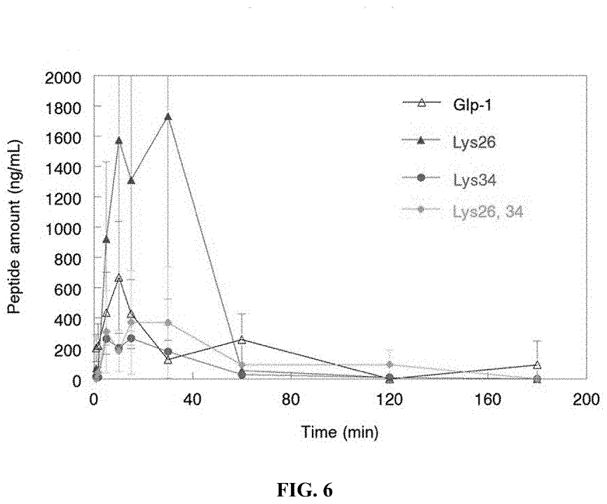

Results: As shown in FIGS. 5A-5B, all ALY688-GLP1v fusion peptides administrated acutely showed a significant lower blood glucose levels after glucose load. ALY688 showed no glucose effect compared with vehicle, while GLP1v (with Gly8 substitution) and exenatide both showed the expected glucose-lowering activity. All four ALY688-GLP1v fusion peptides (same as those in Examples 2-3) also demonstrated glucose lowering actions at least comparable to GLP1v with Lys26 and Lys34 substituted fusion peptides showing greater glucose lowering action compared with GLP1v alone. Thus, the glucose-lowering actions of GLP1 activation were preserved and improved with the administration of ALY688-GLP1v fusion peptides.

### Example 5. Pharmacokinetic Profile of GLP1v and Fusion Peptides in Mice

In order to assess whether ALY688-GLP1v fusion peptides demonstrate differences in pharmacokinetic behavior as a result of being covalently attached, the pharmacokinetic profile of three ALY688-GLP1v fusion peptides was compared with GLP1v peptide.

GLP1v peptide (with Gly8 substitution) and GLP1v fusion peptides were administered once by subcutaneous injection (concentration=10 mg/kg). Blood was collected into K2EDTA tubes containing a cocktails of protease inhibitors (DPP-4 and Aprotinin). The Bioanalytical Method BAM.0634.01 was used for the quantitation of GLP1v and GLP1v fusion peptides in K2EDTA mouse plasma. It is based on protein precipitation extraction followed by LC-MS/MS instrumental analysis and covers a measurement range from 1.00 to 1000 ng/mL. Samples were analyzed on a Waters Acquity liquid chromatograph interfaced with a Thermo Scientific TSQ Vantage triple quadrupole mass spectrometer with ESI ionization.

Results: as shown in FIG. 6 (data from which are also summarized in the table below), significant differences were observed in the exposure following a single injection of each peptide, with ALY688-GLP1v_{(Lys26)} fusion peptide showing a two-fold increase in AUC and an increase of the Tmax from 5 to 10 minutes, while ALY688-GLP1v_{(Lys34)}, and ALY688-GLP1v_{(Lys26,34)} both demonstrated a decrease in total exposure, but with a further increase in the Tmax, indicating a delay in the absorption and extension of the blood levels.

| | Cmax | Tmax | AUC₀₋ₜ |
|---|---|---|---|
| Treatment | (ng/mL) | (min) | (ng*min/mL) |
| GLP1v with Gly8 substitution | 781 | 5 | 31500 |
| ALY688-GLP1v_{(Lys26)} | 1610 | 10 | 61100 |
| ALY688-GLP1v_{(Lys34)} | 267 | 15 | 10600 |
| ALY688-GLP1v_{(Lys26,34)} | 372 | 15 | 21500 |

The sequence of ALY688-GLP1v_{(Lys26,34)} is as follows:

H-His⁷-Gly-Glu-Gly-Thr-Phe-Thr-Ser-Asp-Val-Ser-
Ser-Tyr-Leu-Glu-Gly-Gln-Ala-Ala-Lys²⁶(Gly-Gly-
Gly-DSer-Ala-Phe-DSer-Tyr-Leu-Nva-Pro-Ile-DAsn-
H)-Glu-Phe-Ile-Ala-Trp-Leu-Val-Lys³⁴(Gly-Gly-
Gly-DSer-Ala-Phe-DSer-Tyr-Leu-Nva-Pro-Ile-DAsn-
H)-Gly-Arg³⁶-NH₂

Although the above invention has been described in some detail by way of illustration and example for purposes of clarity of understanding, it is apparent to those skilled in the art that certain changes and modifications will be practiced. Therefore, the description and examples should not be construed as limiting the scope of the disclosed invention.

### SEQUENCE LISTING

(GLP1, or GLP1(7-36) amide)
SEQ ID NO: 1

| | | |
|---|---|---|
| 7 | H-His-Ala-Glu-Gly-Thr-Phe-Thr-Ser-Asp-Val- | 19 |
| | Ser-Ser-Tyr- | |
| 20 | Leu-Glu-Gly-Gln-Ala-Ala-Lys-Glu-Phe-Ile- | 32 |
| | Ala-Trp-Leu- | |
| 33 | Val-Lys-Gly-Arg-NH₂ | 36 |

(ALY688)
SEQ ID NO: 2
H-DAsn-Ile-Pro-Nva-Leu-Tyr-DSer-Phe-Ala-DSer-NH2
SEQ ID NO: 3
H-Lys-Phe-His-Cys-Asn-Ile-Pro-Gly-Leu-Tyr-Tyr-
Phe-Ala-Tyr-His-Ile-Thr-Val-NH₂
SEQ ID NO: 4
H-His-Gly⁸-Glu-Gly-Thr-Phe-Thr-Ser-Asp-Val-Ser-
Ser¹⁸-Tyr-Leu-Glu-Gly22-Gln-Ala-Ala-Lys²⁶-Glu-
IPhe-le-Ala-Trp-Leu-Val-Lys³⁴-Gly-Arg-NH₂
SEQ ID NO: 5
H-His-Gly⁸-Glu-Gly-Thr-Phe-Thr-Ser-Asp-Val-Ser-
Lys¹⁸-Tyr-Leu-Glu-Gly22-Gln-Ala-Ala-Lys²⁶-Glu-
Phe-Ile-Ala-Trp-Leu-Val-Lys³⁴-Gly-Arg-NH₂
SEQ ID NO: 6
H-His-Gly⁸-Glu-Gly-Thr-Phe-Thr-Ser-Asp-Val-Ser-
Ser¹⁸-Tyr-Leu-Glu-Lys²²-Gln-Ala-Ala-Lys²⁶-Glu-
Phe-Ile-Ala-Trp-Leu-Val-Lys³⁴-Gly-Arg-NH₂

1. A fusion peptide comprising a GLP1 variant and at least one adiponectin agonist peptide, wherein the at least one adiponectin agonist peptide is chemically attached to the GLP1 variant via by a spacer.

2. The fusion peptide of claim 1, wherein the GLP1 variant comprises a substitution with Gly at position 8, wherein the position corresponds to the position of SEQ ID NO:1.

3. The fusion peptide of claim 1, wherein the GLP1 variant comprises a substitution with Lys at position 18, wherein the position corresponds to the position of SEQ ID NO:1.

4. The fusion peptide of claim 1, wherein the GLP1 variant comprises a substitution with Lys at position 22, wherein the position corresponds to the position of SEQ ID NO:1.

5. The fusion peptide of any the foregoing claims, wherein the at least one adiponectin agonist peptide is attached via the spacer at the position 26 of the GLP1 variant.

6. The fusion peptide of any of claims 1-4, wherein the at least one adiponectin agonist peptide is attached via the spacer at the position 34 of the GLP1 variant.

7. The fusion peptide of any of claims 1-4, wherein the at least one adiponectin agonist peptide comprises a first adiponectin agonist peptide and a second adiponectin agonist peptide, the first adiponectin agonist peptide and the second adiponectin agonist peptide being the same or different and each attached via a spacer at two different positions of the GLP1 variant.

8. The fusion peptide of claim 7, wherein the different two attachment sites comprise position 26 and position 34 of the GLP1 variant, wherein the positions correspond to the positions of SEQ ID NO:1.

9. The fusion peptide of any of the foregoing claims, wherein the at least one adiponectin agonist peptide comprises ALY688.

10. The fusion peptide of any of the foregoing claims, wherein the spacer comprises GGG.

11. The fusion peptide of claim 1, wherein the GLP1 variant has a sequence selected from the group consisting of SEQ ID NO:4, SEQ ID NO:5, and SEQ ID NO:6.

12. A method of treating a patient having type 2 diabetes mellitus, the method comprising administering to the patient an effective amount of a fusion peptide of any of the foregoing claims.


SEQUENCE LISTING (6 sequences)

SEQ ID NO: 1 - protein, 30 aa, Artificial Sequence
  Synthetic
  MOD_RES (30)..(30): The C-terminal amino acid at position 30 is optionally amidated
HAEGTFTSDVSSYLEGQAAKEFIAWLVKGR

SEQ ID NO: 2 - protein, 10 aa, Artificial Sequence
  Synthetic
  MISC_FEATURE (1)..(1): Xaa at position 1 is D-Asn
  MISC_FEATURE (4)..(4): Xaa at position 4 is Nva
  MISC_FEATURE (7)..(7): Xaa at position 7 is D-Ser
  MISC_FEATURE (10)..(10): Xaa at position 10 is D-Ser-NH2
XIPXLYXFAX

SEQ ID NO: 3 - protein, 18 aa, Artificial Sequence
  Synthetic
  MOD_RES (18)..(18): The C-terminal amino acid at position 18 is optionally amidated
KFHCNIPGLYYFAYHITV

SEQ ID NO: 4 - protein, 30 aa, Artificial Sequence
  Synthetic
  MOD_RES (20)..(20): The amino acid  Lys at position 20 is optionally modified at epsilon amino group
  MOD_RES (28)..(28): The amino acid  Lys at position 28 is optionally modified at epsilon amino group
  MOD_RES (30)..(30): The C-terminal amino acid at position 30 is optionally amidated
HGEGTFTSDVSSYLEGQAAKEFIAWLVKGR

SEQ ID NO: 5 - protein, 30 aa, Artificial Sequence
  Synthetic
  MOD_RES (12)..(12): The amino acid  Lys at position 12 is optionally modified at epsilon amino group
  MOD_RES (30)..(30): The C-terminal amino acid at position 30 is optionally amidated
HGEGTFTSDVSKYLEGQAAKEFIAWLVKGR

SEQ ID NO: 6 - protein, 30 aa, Artificial Sequence
  Synthetic
  MOD_RES (16)..(16): The amino acid  Lys at position 16 is optionally modified at epsilon amino group
  MOD_RES (30)..(30): The C-terminal amino acid at position 30 is optionally amidated
HGEGTFTSDVSSYLEKQAAKEFIAWLVKGR